(19) United States
(12) Patent Application Publication
(10) Pub. No.: US 2026/0294992 A1
(43) Pub. Date: Oct. 1, 2026

(54) METHOD FOR ENHANCING CANNABINOID CONTENT IN CANNABIS LEAVES

(71) Applicant: BIOsp Korea Co., Ltd., Seoul (KR)

(72) Inventors: Noshin Myung, Seoul (KR); Seongjin Park, Seoul (KR)

(21) Appl. No.: 19/093,209

(22) Filed: Mar. 27, 2025

(51) Int. Cl. A61K 36/185

(52) U.S. Cl. CPC A61K 36/3482; A61K 2236/331; A61K 2236/37; A61K 2236/39; A61K 2236/51

(57) ABSTRACT

The present invention relates to a method for enhancing cannabinoid content in cannabis leaves through repeated steam-cooking and cooling cycles, followed by freeze-drying. This process significantly increases cannabinoid concentrations, making previously underutilized cannabis leaves commercially viable for pharmaceutical, nutraceutical, and dietary applications.

### BACKGROUND

### Technical Field

The present invention relates generally to methods for enhancing the cannabinoid content of cannabis leaves. More particularly, the present invention relates to methods involving repeated steam-cooking and cooling cycles followed by freeze-drying, resulting in cannabis leaf compositions with significantly improved cannabinoid concentrations.

### Background of the Invention

Cannabis has traditionally been valued for its diverse array of bioactive cannabinoids, which provide various medicinal, pharmaceutical, and nutritional benefits. Historically, the primary focus of cannabis processing has centered on extracting cannabinoids from cannabis flowers, primarily due to their higher cannabinoid content compared to cannabis leaves. Conversely, fresh cannabis leaves, particularly small leaves containing low cannabinoid concentrations, have been underutilized due to their limited commercial viability and difficulties associated with processing.

Conventional processing techniques predominantly involve extracting cannabinoids into oil form using solvent-based methods. However, these methods are generally unsuitable for processing fresh cannabis leaves, especially those previously considered commercially unusable due to their inherently low cannabinoid levels. Solvent-based extraction techniques require substantial amounts of raw material, involve complex processing equipment, and often result in loss or degradation of heat-sensitive cannabinoids, as well as the introduction of undesired solvent residues.

Furthermore, conventional processing methods have not provided efficient solutions for effectively enhancing the cannabinoid content of fresh cannabis leaves in their natural, unextracted state. Consequently, there remains a need in the industry for innovative processing technologies capable of significantly increasing the cannabinoid concentrations in previously underutilized fresh cannabis leaves, enabling their direct consumption or incorporation into various commercial products without reliance on traditional solvent-based extraction processes.

### SUMMARY

### Technical Problem

The technical problem addressed by the present invention is to overcome limitations associated with conventional extraction techniques and to develop an efficient processing method that can significantly increase cannabinoid concentrations in cannabis leaves, particularly previously underutilized leaves with inherently low cannabinoid content

### Technical Solution

The present invention relates to a novel method specifically designed to enhance the cannabinoid content of cannabis leaves through an optimized sequential treatment comprising steam-cooking, cooling, and freeze-drying processes.

Conventional extraction or processing methods of cannabis leaves often result in low cannabinoid yields, limited bioavailability, and the potential degradation of sensitive cannabinoid compounds due to prolonged exposure to high temperatures or inefficient drying methods. The method disclosed herein addresses these limitations effectively by employing carefully controlled thermal and freeze-drying conditions, thereby significantly increasing cannabinoid concentrations.

According to an exemplary embodiment of the present invention, cannabis leaves undergo an initial steam-cooking process. The steam-cooking step is carried out at a temperature sufficient to effectively activate and increase cannabinoid concentration, preferably ranging from about 50° C. to about 120° C., and most optimally performed at atmospheric pressure conditions. The duration of the steam-cooking step is preferably within the range of about 10 to 120 minutes, more preferably between 10 to 50 minutes, to optimize the cannabinoid content enhancement without degrading heat-sensitive compounds.

Following steam-cooking, the cannabis leaves are subjected to a cooling step at atmospheric pressure for a duration ranging from about 5 to 120 minutes. Preferably, the cooling duration is approximately 10 to 50 minutes. Cooling allows cannabinoids to stabilize, thus avoiding thermal degradation and facilitating additional enhancements in cannabinoid concentration during subsequent processing cycles.

In a preferred embodiment, the steam-cooking step is carried out at atmospheric pressure conditions (approximately 1 atm), thus avoiding complicated pressure control equipment and minimizing thermal degradation risks associated with higher-pressure treatments.

To maximize cannabinoid enhancement, the steam-cooking and cooling processes are repeated sequentially for multiple cycles. According to a preferred embodiment, these steps are repeated sequentially at least two times. Most advantageously, 3 to 4 cycles are employed, as experimental data indicates significant incremental cannabinoid enhancement under these optimized conditions.

Subsequent to the sequential steam-cooking and cooling cycles, the cannabis leaves are subjected to a freeze-drying step. In a preferred embodiment, freeze-drying is performed by initially freezing the treated cannabis leaves at approximately −30° C. and then drying under vacuum conditions at approximately 30° C. Such specific freeze-drying conditions ensure minimal loss of cannabinoids while maintaining the integrity and bioavailability of the enhanced cannabinoids.

Preferably, cannabis leaves selected for processing according to the present invention are characterized by having a length of approximately 1 to 10 cm (not limited thereto), and an initial cannabinoid content ranging from approximately 5 to 20 mg/g. Experimental evidence has shown that leaves within these parameters respond optimally to the described enhancement processes.

Through repeated experimentation, it has been established that cannabinoid content can be significantly increased through 2 or more sequential cycles of steam-cooking and cooling, with the most beneficial results typically observed after 3 to 4 cycles. Specifically, experimental data confirm that cannabinoid content increases by at least approximately 50% when cannabis leaves are processed according to the present method, compared to untreated leaves.

In conclusion, the method of the present invention successfully provides cannabis leaf compositions with significantly enhanced cannabinoid concentrations suitable for direct consumption or further utilization in pharmaceutical products, nutraceutical capsules, tablets, or beverage-compatible capsules. Such enhanced compositions provide improved commercial viability and broadened applicability, including pharmaceutical and nutraceutical applications.

### BRIEF DESCRIPTION OF THE DRAWINGS

FIG. 1 is a flowchart illustrating a method for enhancing cannabinoid content in cannabis leaves according to an embodiment of the present invention.

FIG. 2 shows representative photographic images of cannabis leaves before and after processing according to the method of the present invention, wherein the image on the left represents fresh, untreated cannabis leaves, and the image on the right represents cannabis leaves after undergoing repeated steam-cooking, cooling, and freeze-drying processes, demonstrating physical changes resulting from the processing.

FIGS. 3A to 3C show representative HPLC analysis results of cannabinoid content at different processing stages (raw, 1 cycle, and 2 cycles).”

### DETAILED DESCRIPTION

Hereinafter, preferred embodiments of the present disclosure will be described in detail with reference to the accompanying drawings. Advantages and features of the present invention, and methods of achieving them, will become apparent with reference to the embodiments described below in detail along with the accompanying drawings. However, the technical spirit of the present invention is not limited to the following embodiments but can be implemented in various different forms, and the following embodiments are provided to make the technical spirit of the present invention complete and to fully convey the scope of the present invention to those skilled in the technical field to which the present invention pertains. The technical spirit of the present invention is only defined by the scope of the claims.

In describing the present disclosure, detailed descriptions of related known structures or functions are omitted if it is determined that they may obscure the gist of the present invention.

Unless otherwise defined, the terms used in the following embodiments (including technical and scientific terms) can be used in meanings commonly understood by those skilled in the art to which the present disclosure belongs. However, these terms may vary depending on the intention or precedent of those skilled in the relevant field, or the emergence of new technologies. The terms used in the present disclosure are for the purpose of describing the embodiments and are not intended to limit the scope of the present disclosure.

Singular expressions used in the following embodiments include plural concepts unless they are clearly specified as singular in context. Similarly, plural expressions include singular concepts unless they are clearly specified as plural in context.

In addition, the terms first, second, A, B, (a), (b), etc. used in the following embodiments are only used to distinguish one component from another, and the nature, order, or sequence of those components are not limited by these terms.

Hereinafter, various embodiments of the present disclosure will be described in detail with reference to the accompanying drawings.

The method for enhancing cannabinoid content according to the present invention comprises the steps of steam-cooking cannabis leaves, followed by cooling the steam-cooked leaves, repeating these steam-cooking and cooling cycles sequentially at least two times, and finally, freeze-drying the treated cannabis leaves.

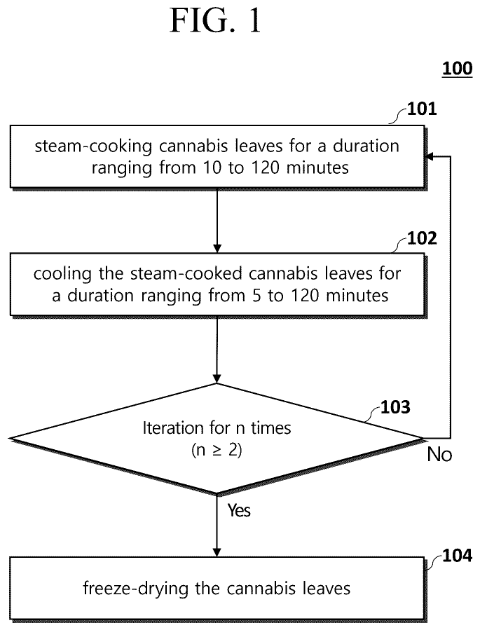

FIG. 1 illustrates a process flowchart showing a method 100 for enhancing cannabinoid content in cannabis leaves according to an embodiment of the present invention.

Referring to FIG. 1, the method 100 includes a step 101 of steam-cooking cannabis leaves. In step 101, the cannabis leaves are steam-cooked for a duration ranging from about 10 to about 120 minutes. Preferably, steam-cooking is performed at atmospheric pressure and at a temperature ranging from about 50° C. to about 120° C., more preferably from about 50° C. to about 90° C.

Subsequently, the steam-cooked cannabis leaves undergo a cooling step 102. In step 102, the cannabis leaves are cooled for a duration ranging from about 5 to about 120 minutes. The cooling step allows stabilization of cannabinoids and prevents degradation during subsequent steam-cooking cycles.

After completion of the cooling step 102, the method proceeds to step 103, where it is determined whether the steam-cooking 101 and cooling 102 steps have been sequentially repeated for at least two cycles (n≥2). If the cycles have not yet reached the predetermined number of iterations (n), the process returns to step 101, repeating the steam-cooking and cooling cycle. Preferably, the cycles are repeated for 3 to 4 times for optimal cannabinoid content enhancement.

Upon completion of the repeated steam-cooking and cooling cycles, the method proceeds to step 104, where the processed cannabis leaves are freeze-dried. The freeze-drying step preferably includes freezing at approximately −30° C. and subsequently drying under vacuum conditions at approximately 30° C. This ensures minimal cannabinoid degradation and preservation of the enhanced cannabinoid content.

### Preparation Step

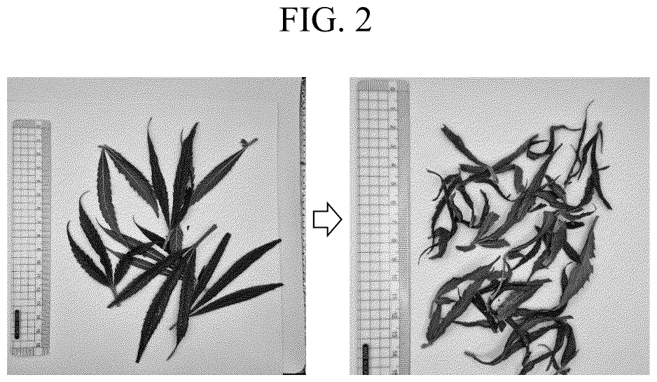

Fresh cannabis leaves suitable for the method typically have lengths ranging from about 1 cm to about 10 cm (FIG. 2). However, the leaf length is not limited to this range, and larger sizes of fresh cannabis leaves may also be utilized in the described method without departing from the scope and spirit of the invention. The cannabinoid content of the cannabis leaves initially ranges from approximately 5 to 20 mg/g. By applying the present inventive method, cannabinoid concentrations within the cannabis leaves can be significantly increased, preferably by at least 50%, relative to untreated cannabis leaves.

### Steam-Cooking and Cooling Steps

The steam-cooking step involves heating cannabis leaves using steam at temperatures ranging from about 50° C. to about 120° C. Preferably, the steam-cooking is conducted at atmospheric pressure (about 1 atm). Optimal results are obtained by performing the steam-cooking step at a temperature range of approximately 50-90° C., although broader temperature ranges can still yield beneficial results. The duration for the steam-cooking step typically ranges from about 10 minutes to about 120 minutes, with optimal results achieved at durations ranging from 10 to 50 minutes.

Following the steam-cooking, the cannabis leaves are cooled at atmospheric pressure for a duration ranging from 5 to 120 minutes. Preferably, the cooling step duration is 10 to 50 minutes. Cooling facilitates stabilization of cannabinoids and prevents thermal degradation during subsequent heating cycles.

The sequential steam-cooking and cooling cycles are repeated multiple times, preferably 3 to 4 cycles, to maximize cannabinoid enhancement. Experimental results have demonstrated that performing these steps repeatedly significantly increases the cannabinoid content of cannabis leaves.

The steam-cooking process facilitates decarboxylation of acidic cannabinoids such as THCA and CBDA, leading to an increase in the bioavailable forms of THC and CBD.

In a preferred embodiment, the steam-cooking step is carried out at atmospheric pressure conditions (approximately 1 atm), thus avoiding complicated pressure control equipment and minimizing thermal degradation risks associated with higher-pressure treatments. Furthermore, controlled thermal processing at atmospheric pressure minimizes cannabinoid degradation while enhancing total cannabinoid concentration through cellular structural modification.

### Freeze-Drying Step

After completing the repeated steam-cooking and cooling cycles, the cannabis leaves are subjected to freeze-drying. Freeze-drying is conducted by initially freezing the cannabis leaves at approximately −30° C. and subsequently drying the frozen cannabis leaves under vacuum at approximately 30° C. Preferably, commercially available freeze dryers such as the Harvest Right freeze dryer (Harvest Right, USA) are used to perform freeze-drying according to manufacturer-recommended settings.

The freeze-drying process removes moisture from the cannabis leaves without applying excessive heat, thereby preserving cannabinoids in their optimal state and minimizing degradation.

The inventive processing method consistently achieves significant cannabinoid enhancement. Particularly, the cannabinoid content of cannabis leaves processed according to the inventive method increases by at least 50% compared to untreated leaves, rendering previously underutilized leaves commercially valuable.

Freeze-drying is chosen over conventional drying methods because it preserves cannabinoid integrity, prevents oxidation, and maintains the bioavailability of heat-sensitive compounds. Unlike air-drying or oven-drying, freeze-drying ensures minimal cannabinoid loss and retains the natural structure of cannabis leaves.

### Preferred Conditions

In a preferred embodiment, the cannabis leaves are steam-cooked and cooled for a total of 3 to 4 cycles, as experimental data indicates that cannabinoid concentration significantly increases within this range. Such optimal processing conditions are as follows:
Initial cannabinoid content: approximately 5 to 20 mg/g
Steam-cooking temperature: approximately 50-90° C.
Steam-cooking duration: approximately 10-50 minutes per cycle
Cooling duration: approximately 10-50 minutes per cycle
Number of cycles: preferably 3-4 cycles
Freeze-drying conditions: freezing at approximately −30° C. followed by drying under vacuum at approximately 30° C.

### Effect of the Invention

Experimental data confirmed through analytical testing in both the United States and Thailand demonstrates the efficacy of the present invention. Specifically, repeated steam-cooking and cooling cycles followed by freeze-drying resulted in cannabinoid concentrations increasing by more than 50% compared to untreated or conventionally treated cannabis leaves. This significant enhancement makes previously underutilized cannabis leaves commercially viable and suitable for diverse applications, including pharmaceuticals, nutraceutical products, dietary supplements, and health-related consumable products.

### Experiment 1

Experiment 1 was conducted from March to April 2022 in Wilson, Oklahoma, USA, with cannabinoid content analyses performed by BUD'S TESTING (Duncan, Oklahoma) using high-performance liquid chromatography (HPLC). The objective of these experiments was to demonstrate the enhancement in cannabinoid content following sequential processing steps according to the inventive method. In Experiment 1.

TABLE 1

| | Total THC | Total THC | | Δ9- | |
|---|---|---|---|---|---|
| Process | (%) | (mg/g) | THCa | THC | INCRS |
| Raw | 1.609% | 16.09 | 1.390% | 0.390% | — |
| 1 cycle | 2.168% | 21.68 | 0.830% | 1.440% | 135% |
| 2 cycles | 2.593% | 25.93 | 0.630% | 2.040% | 161% |

As shown in Table 1, the total THC content increased progressively with each processing cycle. In the raw cannabis leaves (i.e., freeze-dried only without any prior steam-cooking or cooling treatment), the total THC content was measured at 1.609% (16.09 mg/g), with THCa constituting 1.390% and Δ9-THC at 0.390%. After one processing cycle, the total THC content increased to 2.168% (21.68 mg/g), with a reduction in THCa to 0.830% and a corresponding increase in Δ9-THC to 1.440%, indicating partial decarboxylation of THCa. Following two processing cycles, the total THC content further increased to 2.593% (25.93 mg/g), with THCa decreasing to 0.630% and Δ9-THC rising to 2.040%. The increase rate (INCRS) compared to the raw state was recorded at 135% after one cycle and 161% after two cycles. These results demonstrate that repeated steam-cooking and cooling cycles, followed by freeze-drying, effectively enhance cannabinoid content in cannabis leaves by promoting THCa decarboxylation and increasing the concentration of bioavailable Δ9-THC.

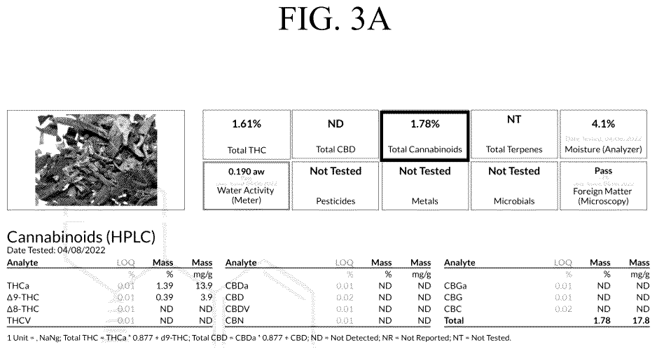

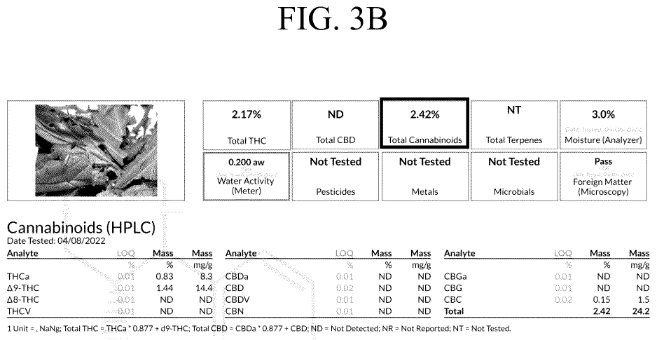

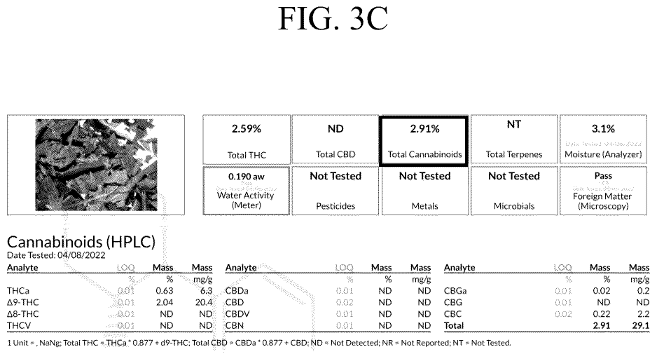

FIGS. 3A to 3C illustrate representative HPLC analysis results for cannabis leaves in various processing states:

FIG. 3A shows the HPLC analysis data for raw cannabis leaves (i.e., freeze-dried only without any prior steam-cooking or cooling treatment), wherein the initial total cannabinoid content measured was approximately 1.78% (17.8 mg/g). The corresponding total THC content was 1.61%.

FIG. 3B presents the HPLC analysis data for cannabis leaves after undergoing one processing cycle (steam-cooking and cooling). The total cannabinoid content increased to approximately 2.42% (24.2 mg/g), and the total THC content also rose to 2.17%, demonstrating a substantial improvement relative to the raw state.

FIG. 3C depicts the HPLC analysis data for cannabis leaves subjected to two processing cycles (two sequential steam-cooking and cooling cycles). At this stage, the total cannabinoid content further increased to approximately 2.91% (29.1 mg/g), while total THC content reached 2.59%, clearly showing cumulative enhancement in both total cannabinoid and total THC levels as the number of cycles increased.

These results clearly confirm that the inventive processing method significantly enhances cannabinoid content. Specifically, the cannabinoid content increased from an initial 1.78% (raw) to 2.91% after two processing cycles, representing a substantial cumulative increase in cannabinoid concentration.

### Experiment 2

The following additional experiments were conducted by Eastern Spectrum Group Ltd. in Thailand, as part of a research and development program carried out from November 2024 to January 2025. As shown in the experimental results summarized in Table 2, cannabinoid content, including total THC, Δ9-THC, and THCa, significantly increases through repeated steam-cooking and cooling cycles:

TABLE 2

| | Total THC | Total THC | | Δ9- | |
|---|---|---|---|---|---|
| Process | (%) | (mg/g) | THCa | THC | INCRS |
| Raw | 1.259% | 12.59 | 1.379% | 0.050% | — |
| 1 cycle | 2.320% | 23.20 | 2.499% | 0.128% | 184% |
| 2 cycles | 1.961% | 19.61 | 1.986% | 0.219% | 156% |
| 3 cycles | 2.366% | 23.66 | 2.343% | 0.311% | 188% |
| 4 cycles | 1.919% | 19.19 | 1.709% | 0.420% | 152% |
| 5 cycles | 1.843% | 18.43 | 1.543% | 0.490% | 146% |
| 6 cycles | 1.404% | 14.04 | 1.022% | 0.508% | 112% |

As demonstrated by the experimental data (Table 2), cannabinoid levels, including total THC, Δ9-THC, and THCa, were enhanced through repeated cycles of steam-cooking and cooling. Specifically, the total THC content increased substantially after repeated processing cycles, reaching a peak enhancement after 3 to 4 cycles.

As shown in Table 2, the total THC content in raw cannabis leaves (i.e., freeze-dried only without any prior steam-cooking or cooling treatment) was initially measured at 1.259% (12.59 mg/g), with THCa constituting 1.379% and Δ9-THC at 0.050%.

After one processing cycle, the total THC content increased to 2.320% (23.20 mg/g), with THCa rising to 2.499% and Δ9-THC increasing to 0.128%, resulting in an increase rate (INCRS) of 184%. Following two cycles, the total THC content slightly decreased to 1.961% (19.61 mg/g), while THCa remained at 1.986% and Δ9-THC increased to 0.219%, maintaining a high increase rate of 156%.

At three cycles, the total THC content peaked at 2.366% (23.66 mg/g), with THCa measured at 2.343% and Δ9-THC at 0.311%, representing the highest increase rate of 188%.

Beyond three cycles, the total THC content showed a declining trend, decreasing to 1.919% (19.19 mg/g) at four cycles and further decreasing to 1.843% (18.43 mg/g) and 1.404% (14.04 mg/g) at five and six cycles, respectively. In this sense, performing more than four cycles demonstrated diminishing returns, with cannabinoid content slightly decreasing due to potential thermal degradation or compound loss.

### Experiment 3

The following additional experiment 3 was conducted by Eastern Spectrum Group Ltd. in Thailand, as part of a research and development program carried out from November 2024 to January 2025. These experiments were performed independently from the experimental data shown previously in Table 2, utilizing separate cannabis leaf samples and analytical conditions.

Cannabis leaves were processed according to the inventive method comprising repeated cycles of steam-cooking and cooling. Total THC content was quantitatively measured after each iteration to confirm cannabinoid enhancement, as summarized in Table 3 below.

TABLE 3

| | Total THC | Total THC | | Δ9- | |
|---|---|---|---|---|---|
| Process | (%) | (mg/g) | THCa | THC | INCRS |
| Raw | 1.035% | 10.35 | 1.163% | 0.015% | — |
| 1 cycle | 1.174% | 11.74 | 1.306% | 0.029% | 113% |
| 2 cycles | 1.474% | 14.74 | 1.615% | 0.058% | 142% |
| 3 cycles | 1.795% | 17.95 | 1.887% | 0.140% | 173% |
| 4 cycles | 2.294% | 22.94 | 2.210% | 0.355% | 222% |
| 5 cycles | 1.726% | 17.26 | 1.506% | 0.405% | 167% |
| 6 cycles | 1.653% | 16.53 | 1.097% | 0.691% | 160% |

As shown in Table 3, the total THC content in raw cannabis leaves (i.e., freeze-dried only without any prior steam-cooking or cooling treatment) was initially measured at 1.035% (10.35 mg/g), with THCa at 1.163% and Δ9-THC at 0.015%. After one processing cycle, the total THC content increased to 1.174% (11.74 mg/g), with THCa rising to 1.306% and Δ9-THC increasing to 0.029%, resulting in an increase rate (INCRS) of 113%.

Following two cycles, the total THC content increased further to 1.474% (14.74 mg/g), with THCa at 1.615% and Δ9-THC at 0.058%, corresponding to an increase rate of 142%. At three cycles, the total THC content reached 1.795% (17.95 mg/g), with THCa increasing to 1.887% and Δ9-THC significantly rising to 0.140%, marking an increase rate of 173%.

The highest total THC content was recorded after four processing cycles, reaching 2.294% (22.94 mg/g), with THCa at 2.210% and Δ9-THC increasing to 0.355%, representing a maximum increase rate of 222%. However, beyond four cycles, the total THC content began to decline, decreasing to 1.726% (17.26 mg/g) at five cycles and further reducing to 1.653% (16.53 mg/g) at six cycles.

These results confirm that repeated steam-cooking and cooling cycles enhance cannabinoid content up to a certain threshold, after which excessive processing leads to cannabinoid degradation. Similar to the findings in Table 2, the optimal number of cycles for maximizing cannabinoid enhancement appears to be around three or four cycles, beyond which cannabinoid loss becomes evident.

This experiment conducted by Eastern Spectrum Group Ltd. independently validates the effectiveness of the inventive method in enhancing cannabinoid content, thereby highlighting its applicability in various medicinal, pharmaceutical, and commercial applications.

### BEST MODE

Comprehensively considering the data provided by Table 1 (U.S. experiments), Table 2 (Thailand experiments), and Table 3 (additional Thailand experiments), it has been demonstrated that repeating the steam-cooking and cooling cycles significantly enhances cannabinoid content. Particularly, cannabinoid content notably improves when at least two iterations of processing cycles are performed. However, experimental data consistently indicate that cannabinoid enhancement peaks within approximately three to four processing cycles.

It is noted that different cultivation conditions and cannabis strains were used across the experiments. Specifically, Experiment 1 was conducted using cannabis leaves harvested from indoor-grown plants, whereas Experiments 2 and 3 utilized leaves from outdoor-grown plants. Additionally, the cannabis strains used in each experiment were not identical. Despite these differences in cultivation environment and genetic strain, a consistent trend of enhancement in both total cannabinoid content and total THC levels was observed across all experiments. This consistent outcome demonstrates the broad applicability and robustness of the inventive method in improving cannabinoid concentrations in cannabis leaves, regardless of growth conditions or strain variability.

Experimental results indicate that cannabinoid content increases significantly with multiple cycles, reaching peak enhancement at 3 to 4 cycles. Beyond 4 cycles, cannabinoid levels show a decline, likely due to prolonged thermal exposure leading to degradation. Therefore, the optimal number of iterations (best mode) for maximizing cannabinoid content enhancement according to the inventive method is identified as approximately 3 to 4 cycles.

The embodiments described herein have been presented for illustrative purposes only, and it should be understood that various modifications and changes may be made without departing from the scope and spirit of the present invention as defined by the appended claims. The embodiments described herein are merely illustrative and are not intended to limit the invention in any way. Those skilled in the art will appreciate that numerous modifications, variations, and adaptations may be made to the specific embodiments disclosed herein without departing from the spirit and scope of the invention as defined by the appended claims. The scope of the present invention should be determined solely by reference to the appended claims, rather than by the specific embodiments or examples described above.

What is claimed is:

1. A method for enhancing cannabinoid content in cannabis leaves, comprising:
steam-cooking cannabis leaves for a duration ranging from 10 to 120 minutes;
cooling the steam-cooked cannabis leaves for a duration ranging from 5 to 120 minutes;
repeating the steam-cooking and cooling steps sequentially for at least 2 cycles; and
freeze-drying the cannabis leaves after the completion of the repeated steam-cooking and cooling steps.

2. The method according to claim 1, wherein the cannabis leaves have a cannabinoid content ranging from 5 to 20 mg/g.

3. The method according to claim 1, wherein the steam-cooking step is performed at atmospheric pressure.

4. The method according to claim 1, wherein the steam-cooking step is performed at a temperature ranging from 50° C. to 120° C.

5. The method according to claim 1, wherein the steam-cooking and cooling steps are repeated for 3 to 4 cycles.

6. The method according to claim 1, wherein the freeze-drying step comprises freezing at −30° C. and drying at 30° C. under vacuum.

7. A cannabis leaf composition produced by the method of claim 1, wherein the cannabis leaf composition has an enhanced cannabinoid content that is at least 50% greater than that of untreated cannabis leaves.
